(19) United States
(12) Patent Application Publication
(10) Pub. No.: US 2025/0126416 A1
(43) Pub. Date: Apr. 17, 2025

(54) TEMPERATURE SENSING FOR LEFT-RIGHT DETERMINATION IN HEARING DEVICES

(71) Applicant: Starkey Laboratories, Inc., Eden Prairie, MN (US)

(72) Inventors: Kyle Olson, St Louis Park, MN (US); Zachary J. Burmeister, Chaska, MN (US); John Bradley Etherington, Eagan, MN (US); Andy Shi-Hsuan Lin, Chanhassen, MN (US); Mark J. Schroeder, Eden Prairie, MN (US)

(21) Appl. No.: 18/910,888

(22) Filed: Oct. 9, 2024

Related U.S. Application Data
(60) Provisional application No. 63/590,076, filed on Oct. 13, 2023.

(51) Int. Cl. H04R 25/00; A61B 5/00; A61B 5/01

(52) U.S. Cl. CPC H04R 25/50; A61B 5/01; A61B 5/6817

(57) ABSTRACT

Disclosed herein, among other things, are systems and methods for temperature detection for hearing device applications. A method includes obtaining a first temperature measurement from a first sensor from a first side of a housing of a hearing device, and obtaining a second temperature measurement from a second sensor from a second side of the housing. The first temperature measurement is compared to the second temperature measurement, and a determination is made whether the hearing device is positioned on a right side or a left side of a wearer of the hearing device, based on the comparison. The hearing device is configured as a right device or a left device based on the determination.

### CROSS-REFERENCE TO RELATED APPLICATION

This patent application claims the benefit of U.S. Provisional Patent Application No. 63/590,076, filed Oct. 13, 2023, which is incorporated by reference herein in its entirety.

### TECHNICAL FIELD

This document relates generally to hearing device systems and more particularly to temperature sensing for determining left-right devices for hearing device applications.

### BACKGROUND

Examples of hearing devices, also referred to herein as hearing assistance devices or hearing instruments, include both prescriptive devices and non-prescriptive devices. Specific examples of hearing devices include, but are not limited to, ear-wearable devices such as hearing aids, headphones, assisted listening devices, and earbuds.

Hearing aids are used to assist patients suffering hearing loss by transmitting amplified sounds to ear canals. In one example, a hearing aid is worn in and/or around a patient's ear. Hearing aids may include processors and electronics that improve the listening experience for a specific wearer or in a specific acoustic environment.

Hearing devices may be uniform in appearance and may be adaptable to fit in or on either ear of a wearer. However, certain functions of the hearing device may be dependent upon which ear (left or right) of the wearer the device currently is placed. For example, the device may stream stereo audio that is left-right dependent, or the wearer may have differing hearing loss between ears that requires different gain settings for their left and right ears. Thus, improved systems and methods of determining in which ear of a wearer the device is placed are needed.

### SUMMARY

Disclosed herein, among other things, are systems and methods for temperature sensing for hearing device applications. A method includes obtaining a first temperature measurement from a first sensor from a first side of a housing of a hearing device, and obtaining a second temperature measurement from a second sensor from a second side of the housing. The first temperature measurement is compared to the second temperature measurement, and a determination is made whether the hearing device is positioned on a right side or a left side of a wearer of the hearing device, based on the comparison. The hearing device is configured as a right device or a left device based on the determination.

Various aspects of the present subject matter include a hearing device including a housing, a first sensor on or in a first side of the housing, a second sensor on or in a second side of the housing, and hearing electronics within the housing configured to be connected to a microphone. The hearing electronics include at least one processor and a memory including instructions that, when executed by the at least one processor, cause the at least one processor to perform operations to obtain a first temperature measurement from the first sensor, obtain a second temperature measurement from the second sensor, compare the first temperature measurement to the second temperature measurement, based on the comparison, determine whether the hearing device is positioned on a right side or a left side of a wearer of the hearing device, and configure the hearing device as a right device or a left device based on the determination.

This Summary is an overview of some of the teachings of the present application and not intended to be an exclusive or exhaustive treatment of the present subject matter. Further details about the present subject matter are found in the detailed description and appended claims.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1A illustrates a block diagram of a hearing device including temperature detection, according to various embodiments of the present subject matter.

FIG. 1B illustrates a block diagram of a system for temperature detection and wireless communication for hearing devices, according to various embodiments of the present subject matter.

FIG. 2 illustrates a hearing device including temperature detection, according to various embodiments of the present subject matter.

FIG. 3 illustrates a flow diagram of a method of temperature detection for hearing devices, according to various embodiments of the present subject matter.

FIG. 4 illustrates a block diagram of an example machine upon which any one or more of the techniques discussed herein may perform.

### DETAILED DESCRIPTION

The following detailed description of the present subject matter refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment. The following detailed description is demonstrative and not to be taken in a limiting sense. The scope of the present subject matter is defined by the appended claims, along with the full scope of legal equivalents to which such claims are entitled.

The present detailed description will discuss hearing devices generally, including ear-wearable devices including earbuds, headsets, headphones and hearing assistance devices using the example of hearing aids. Other hearing devices include, but are not limited to, those in this document. It is understood that their use in the description is intended to demonstrate the present subject matter, but not in a limited or exclusive or exhaustive sense.

Hearing devices may be uniform in appearance and may be adaptable to fit in either ear of a wearer. However, certain functions of the hearing device may be dependent upon which ear (left or right) of the wearer the device currently is placed. For example, the device may stream stereo audio that is left-right dependent, or the wearer may have differing hearing loss between ears that requires different gain settings for their left and right ears.

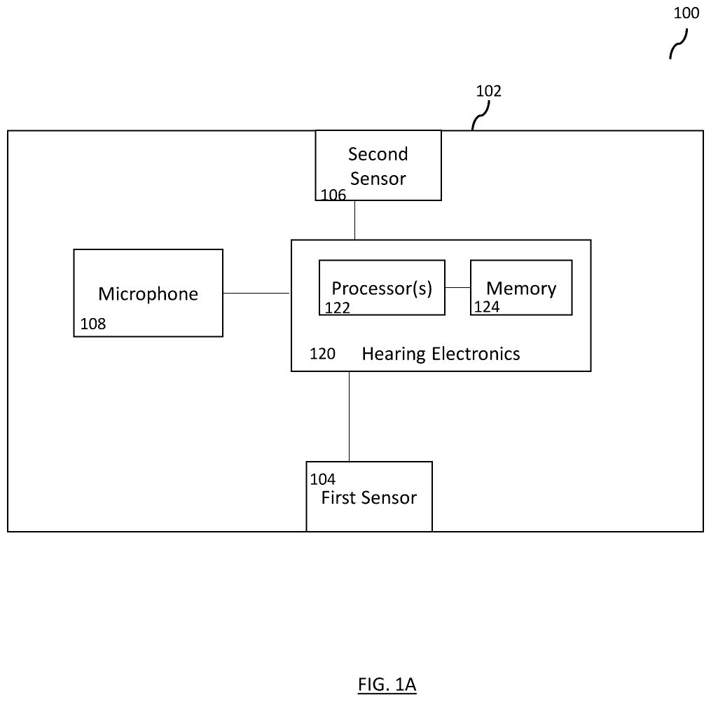

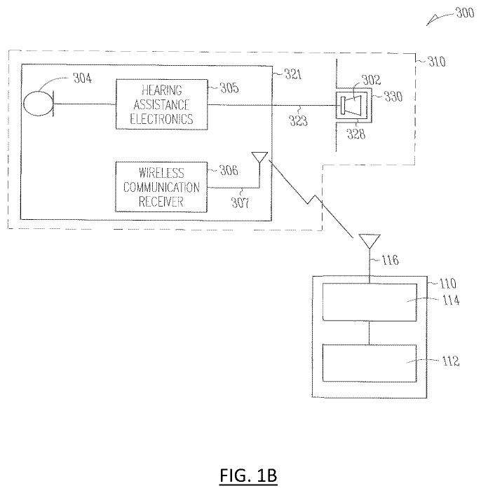

FIG. 1A illustrates a block diagram of a hearing device including temperature detection, according to various embodiments of the present subject matter. The hearing device 100 includes a housing 102, a first sensor 104 on or in a first side of the housing, a second sensor 106 on or in a second side of the housing, and hearing electronics 120 within the housing configured to be connected to a microphone 108. The first side and second side are on opposite sides of the device, in various examples. The hearing electronics 120 include at least one processor 122 and a memory 124 including instructions that, when executed by the at least one processor 122, cause the at least one processor 122 to perform operations to obtain a first temperature measurement from the first sensor 104, obtain a second temperature measurement from the second sensor 106, compare the first temperature measurement to the second temperature measurement, based on the comparison, determine whether the hearing device 100 is positioned on a right side or a left side of a wearer of the hearing device 100, and configure the hearing device 100 as a right device or a left device based on the determination.

In various examples, to configure the hearing device, the processor is configured to determine gain settings for the hearing device based on audiograms for left and right ears of the wearer. The processor is further configured to determine a body temperature of the wearer using the first temperature measurement or the second temperature measurement, in various examples. In various examples, the processor is configured to send a notification to the wearer when the first temperature measurement or the second temperature measurement indicates an abnormal body temperature of the wearer. The notification to the wearer is delivered via a mobile application on a smartphone in communication with the hearing device, in some examples. In some examples, the notification to the wearer includes a message with instructions for reducing temperature in the hearing device. The notification to the wearer is delivered via the hearing device, in some examples. In some examples, the notification to the wearer includes an audible notification. In various examples, the memory includes firmware for the device. The hearing device is a hearing aid, in one example.

FIG. 1B illustrates a block diagram of a system 300 for temperature detection and wireless communication for hearing devices, according to various embodiments of the present subject matter. The system 300 shows an external device 110 in wireless communication with a hearing device 310. In various embodiments, the hearing device 310 includes a first housing 321, an acoustic receiver or speaker 302 in a second housing 328 positioned in or about the ear canal 330 of a wearer and conductors 323 coupling the speaker 302 to the first housing 321 and the electronics enclosed therein. The electronics enclosed in the first housing 321 include a microphone 304, hearing assistance electronics 305, a wireless communication receiver 306 and an antenna 307, in an embodiment. In various embodiments, the hearing assistance electronics 305 includes at least one processor and memory components. The memory components store program instructions for the at least one processor. The program instructions include functions allowing the processor and other components to process audio received by the microphone 304 and transmit processed audio signals to the speaker 302. The speaker 302 emits the processed audio signal as sound in the user's ear canal. In various embodiments, the hearing assistance electronics includes functionality to amplify, filter, limit, condition or a combination thereof, the sounds received using the microphone 304.

In the illustrated embodiment of FIG. 1B, the wireless communication receiver 306 is connected to the hearing assistance electronics 305 and the conductors 323 connect the hearing assistance electronics 305 and the speaker 302. In various embodiments, the external device 110 includes a streaming audio device such as an assistive listening device (ALD), a programmer, a smartphone, a wearable device, a tablet, a personal computer, or other device capable of wireless communication. The external device 110 includes an antenna 116 connected to a radio circuit 114 that include a transmitter, in an embodiment. In various embodiments, the external device 110 includes one or more processors 112 or processing components. The external device 110 may also include one or more microphones and/or one or more speakers, in various embodiments.

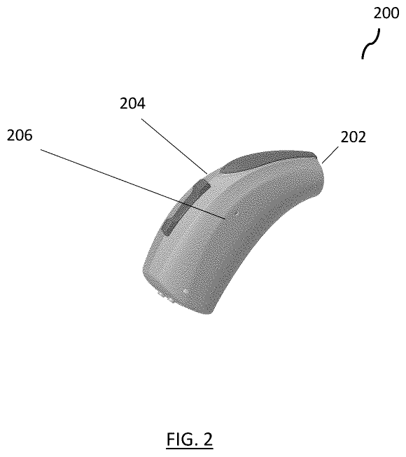

FIG. 2 illustrates a hearing device including temperature detection, according to various embodiments of the present subject matter. The hearing device 200 includes a housing 202, in this case a behind-the-ear housing, with sensors 204, 206 on opposite sides of the housing 202. The sensors 204, 206 may be placed inside or outside the housing, and may be on a surface of the housing in various examples. The sensors may be fixed or adjustable in position, in various examples. In various examples, the sensors 204, 206 may include any type of sensor configured to obtain a temperature measurement.

In various examples, the present system and method is used with devices that have a behind-the-ear housing, such as behind-the-ear (BTE) or receiver-in-canal (RIC) hearing aids. Other types of devices may be used without departing from the scope of the present subject matter. RIC/BTE devices are programed as left/right side devices and fitted with left/right cables. Audiologist can often mistakenly fit a left device with right cable. In some examples, hearing devices with non-left/right specific cables (S-cables) can be used in either ear. However, such hearing devices still have to select which stereo signal to stream to which device, and the device should provide proper gain settings for different audio grams for the left/right ears, so determining the proper ear that the device is placed in is important for these devices.

In some examples, wearers of the device will want different controls on the left/right ear so each device needs to know which side of the head the device is placed on to configure the controls of the device. A standard hearing device may have different microphone locations depending on the side of the head and thus might require slightly different gain settings based on which ear the device is placed in, in some examples. In various examples, the present system may alert users of mistakenly placing devices on the wrong ear. The present system may provide for automatically turning devices on when placed on the ear and automatically turning devices off when removed from the ear by using the present temperature sensing, in various examples. In some examples, the present system may compute the body temperature of the wearer using the temperature sensor(s) behind the ear, and may warn the wearer (using an alert) of an abnormal body temperature. In various examples, the present system may control power circuitry to disconnect the device battery during high temperature events (such as a short circuit).

In various examples, the present subject matter provides two temperature sensors placed within a standard hearing device (e.g., behind the ear housing) such that the first sensor is either on “head side” or the “ambient side” (opposite of the head side) of the device when placed on the ear, and the second sensor is on the opposite side. In most conditions, the “head side” sensor will be a higher temperature than the “ambient side” sensor. For example, a device placed on the left ear would have the first sensor on the ambient side and second on the head side, while if the same device was placed on the right ear, then the first sensor would now be on the head side and the second will be on the ambient side. Since the head side temperature will be higher (in most cases), if the first sensor has a higher temperature than the second sensor then the device is placed on the right side of the head. However, if the first sensor has a lower temperature than the second sensor then the device is placed on the left side of the head, in various examples.

In some situations, the head side temperature is lower than the ambient side, when the ambient temperature is higher than the core body temperature of the user (37 C or 98.6 F). However, it may not be accurate to switch which side is predicted when the temperature sensors are both above 98.6 F, because not every human has the exact same body temperature and each human's body temperature changes throughout the day from many causes (circadian rhythm, disease, eating/drinking, fever, etc.). In another example, the system may ignore the sensor side prediction when both sensors are above ˜95 F and instead rely on the previously known value or a default value. The stability of the temperature sensors should also be considered, in various examples. If the temperature is rising or falling quickly then the devices are changing state and the head side may have the cooler temperature now but will soon have the warmer temperature. For this reason, an extrapolation technique may be used to predict the final temperature of the sensors, or the system may wait a certain time for the sensors to become stable before running the side prediction algorithm, in various examples. The extrapolation technique can be accomplished through fitting a series of exponential decays to the previous several seconds of data then using that model fit to predict the temperature a few minutes in the future, in various examples. The more data and time used in fitting the model allows for further away in time predictions, in various examples. In some examples, the left versus right detection is done within seconds of placing the device on the ear, to enable proper device settings for the user. Other time periods may be used without departing from the scope of the present subject matter.

In various examples, the present temperature sensors can be used with other instant auto on/off sensors (IR proximity, capacitive) so that the system only decides which side (left or right) the devices are being worn on when the devices have been removed and put back on the head. This prevents the device from switching from one side to the other from erroneous signals. In addition, the present system provides for using ear-to-ear communication such that the two devices can agree that they are on opposite sides of the head, in some examples. If both devices sense they are the same side, then a default or previous value can be used to ensure each device selects a different side of the head, in some examples.

According to some examples, the present system only allows side switching detection after a power cycle. However, this would not allow “hot switching,” meaning switching devices' sides after wearing them and with sides already determined. To overcome this case, another example of the present subject matter allows side switching after a dip or a fluctuation of a certain range in temperature was detected as it most likely represents the devices were removed from the body. A dip or fluctuation in temperature could potentially be in the range of 0.5° C. or 1° C. within a period of 5 or 10 seconds, in an example. These extra precautions prevent undesired switching happening during events such as sun exposure, campfire, or other scenarios where external heat source causes the ambient side to temporarily measure above body temperature. When the user moves indoor or into a shade, this temperature fluctuation will trigger the side switching and correct the sides designation if it was different than the previously known setting, in an example.

Additionally, for some examples an inertial measurement unit (IMU) can be used to determine the direction of gravity and confirm the side of the head selection by comparing with the gravity vector of the opposite device. For example, for in-ear devices gravity will point up/down depending on the left/right side insertion but only in typical standing/sitting positions. Depending on the design of the device, if the wearer is either in the prone position (laying on their back) or laying on their side, the IMU would not to help determine orientation.

According to various examples, once the side of the head (right or left) of the device is known, additional features are provided by the present subject matter. In one example, for devices that should not be worn on either side (side-specific devices), a warning/notification (phone app, haptic, audible, etc.) can be presented to the user/wearer to instruct the wearer to switch the devices to opposite ears/sides. In another example, for devices that can be worn on either side, once the side is determined the device can present itself to a wirelessly connected device (e.g., smartphone) as the correct side and receive the correct stereo audio, and send correct side data (health metrics, data logs, etc.) to the smartphone. In various examples, the devices may also have user interface (UI) functions like button presses that behave differently on the left or right side. For example, there may be only one button on each side, and the right button could be set for volume up, and the left button volume down. The present system sets the function of these buttons based on the left/right determination, such that if the user puts the devices on the opposite way each time, they will still have the same side-specific controls and not be confused.

In some examples, for users with different audiograms (different hearing losses) the device can automatically provide the correct gain settings to each ear once the left/right determination is made. For example, each device may have both left and right gains settings programmed into them, and the sensor will select which gain settings to use when the user is wearing the devices based on the left/right determination.

According to various examples, some hearing devices have non-symmetrical microphone locations, so when a device is placed on the left or right side the audio path from an ambient area to the microphone to the receiver to the eardrum may be slightly different on the two sides of the head of the user. This may be due to physical differences in the user's ear, or mechanical design of the device. By knowing which side of the head the device is on, the gain settings can be programmed for optimal sound quality because the audio path is now known, in various examples.

In some examples, to sense the left/right location of the device, the two temperature sensors can be used to calculate the body temperature of the wearer:

$${T}_{b o d y} = \frac{{T}_{h e a d} - {T}_{a m b i ent} - offset}{slop e} + {T}_{h e a d}$$

A similar equation can also be used to calculate the ambient temperature. Since the temperature sensors will be outside of the ear canal in some examples, they will be measuring slightly cooler temperatures than in-the-canal temperature sensors. Because the sensors will be colder, the body temperature estimate will be less accurate than the in-canal sensors, however the estimate for ambient temperature will be more accurate. Additionally, these sensors may be used in conjunction with the in-canal sensors to improve the body temperature estimate further by better compensating for ambient effects on the in-canal temperature sensors.

Using the above equation may also be used to determine which side of the head the devices are placed on, by switching the head and ambient values in some examples. The correct prediction will show a body temperature near 37 C or 98.6 F, but if the head and ambient sensors are switched the T_{body} value will be calculated further away from this value. Since body temperature is usually very stable other than during fevers or hypothermia, this method will be effective even when the temperature sensors are above 95 F. In some examples, even during a fever or hypothermic period the devices should calculate a body temperature near the same value when they have the correct prediction for the side of the head. However, using this equation may be slow to converge on the body temperature, so this calculation may be used as a confirmation (instead of initial prediction) that the system has determined the correct side, in various examples.

In various examples, the calculated body temperature can then be used as a continuously worn thermometer for the user. The device may inform the user of temperature trends throughout the day, and if abnormal temperatures are sensed inform the user of a hyper/hypothermia event, in various examples.

In some examples, digital temperature sensors have threshold crossing interrupts that can be set to control switches on the high-power supply lines. If the temperature of the hearing device rises above a safe limit due to a fault on a high-power supply line, the sensor may shut off the supply line, thus removing the heat source, in various examples. In one example, once the device's temperature decreases to safe levels, the switch will allow the hearing aid to be turned back on. In various examples, for a host to control this supply line under nominal (safe) temperature conditions, the threshold can be set to the lowest possible temperature to force the supply off. If the host wants to turn the switch on, it increases the threshold to the safe limit, and if the temperature is below the safe limit the switch will turn on, but it will turn off if the temperature increases above the safe limit, in various examples. In some examples, LED indicators (e.g., blue and red) can be used to indicate which side is assigned if available on the device.

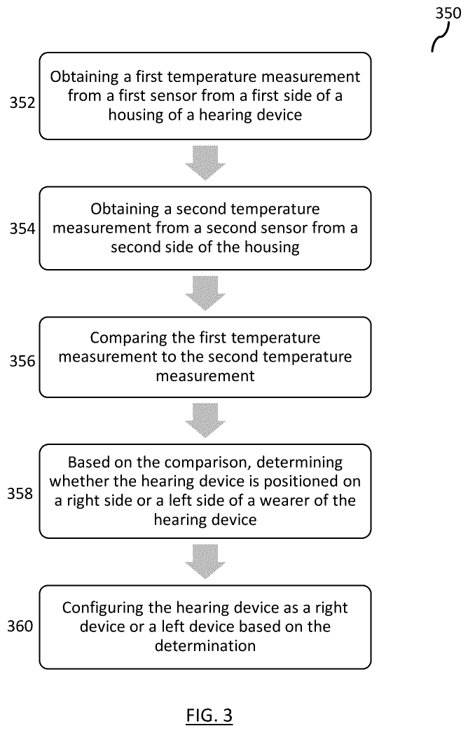

FIG. 3 illustrates a flow diagram of a method of temperature detection for hearing devices, according to various embodiments of the present subject matter. The method 350 includes obtaining a first temperature measurement from a first sensor from a first side of a housing of a hearing device, at step 352, and obtaining a second temperature measurement from a second sensor from a second side of the housing, at step 354. At step 356, the first temperature measurement is compared to the second temperature measurement, and a determination is made whether the hearing device is positioned on a right side or a left side of a wearer of the hearing device based on the comparison, at step 358. At step 360, the hearing device is configured as a right device or a left device based on the determination.

According to various examples, configuring the hearing device includes determining gain settings for the hearing device based on audiograms for left and right ears of the wearer. Configuring the hearing device includes determining a stereo signal to stream to the hearing device, in one example. In some examples, configuring the hearing device includes configuring controls on the housing of the hearing device. The method further includes sending a notification to the wearer when, based on the comparison, the hearing device is placed in a wrong ear of the wearer, in an example. In various examples, the method further includes determining a body temperature of the wearer using the first temperature measurement or the second temperature measurement. In some examples, the method further includes sending a notification to the wearer when the first temperature measurement or the second temperature measurement indicates an abnormal body temperature of the wearer.

The method further includes automatically turning the hearing device on when the first temperature measurement or the second temperature measurement indicates that the hearing device has been placed on an ear of the wearer, in one example. In other examples, the method further includes automatically turning the hearing device off when the first temperature measurement or the second temperature measurement indicates that the hearing device has been removed from an ear of the wearer. In various examples, the method further includes automatically disconnecting a battery of the hearing device when the first temperature measurement or the second temperature measurement indicates a high temperature event above a predetermined temperature threshold.

In various examples, the present system includes software to perform the measurement and the notification. In some examples, the software executes on a processor in the hearing device, such as in firmware of the processor. In other examples, the software may be executed partially or wholly on a device in communication with the hearing device, such as a smartphone. In some example, the present temperature detection scheme can be used to detect temperature in other devices, and is not limited to hearing or ear-wearable devices.

Obtaining a temperature measurement from the sensor within the housing includes obtaining the temperature measurement from a temperature sensor of an inertial measurement unit (IMU) within the housing, in an example. In various examples, sending the notification to the wearer of the hearing device includes sending instructions to the wearer for reducing temperature within the device. Sending the notification to the wearer of the hearing device includes sending an audible notification to the wearer using the hearing device, in an example. In various examples, sending the notification to the wearer of the hearing device includes sending the notification to the wearer of a hearing aid.

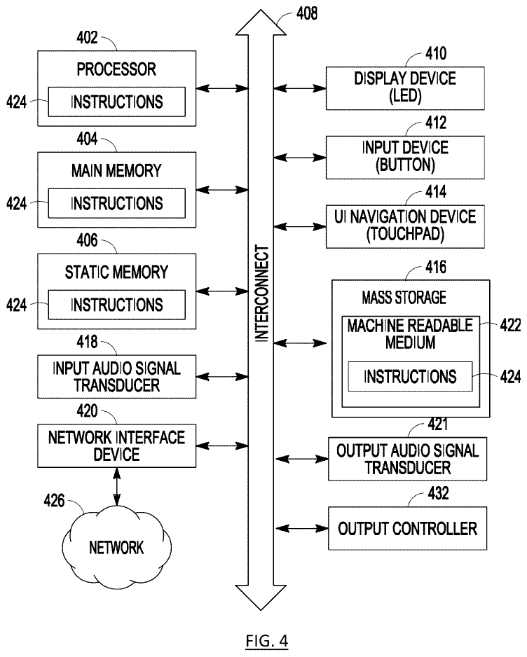

FIG. 4 illustrates a block diagram of an example machine 400 upon which any one or more of the techniques (e.g., methodologies) discussed herein may perform. In alternative embodiments, the machine 400 may operate as a standalone device or may be connected (e.g., networked) to other machines. In a networked deployment, the machine 400 may operate in the capacity of a server machine, a client machine, or both in server-client network environments. In an example, the machine 400 may act as a peer machine in peer-to-peer (P2P) (or other distributed) network environment. The machine 400 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 400 may include a hardware processor 402 (e.g., a central processing unit (CPU), a graphics processing unit (GPU), a hardware processor core, or any combination thereof), a main memory 404 and a static memory 406, some or all of which may communicate with each other via an interlink (e.g., bus) 408. The machine 400 may further include a display unit 410, an alphanumeric input device 412 (e.g., a keyboard), and a user interface (UI) navigation device 414 (e.g., a mouse). In an example, the display unit 410, input device 412 and UI navigation device 414 may be a touch screen display. The machine 400 may additionally include a storage device (e.g., drive unit) 416, one or more input audio signal transducers 418 (e.g., microphone), a network interface device 420, and one or more output audio signal transducer 421 (e.g., speaker). The machine 400 may include an output controller 432, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 416 may include a machine readable medium 422 on which is stored one or more sets of data structures or instructions 424 (e.g., software) embodying or utilized by any one or more of the techniques or functions described herein. The instructions 424 may also reside, completely or at least partially, within the main memory 404, within static memory 406, or within the hardware processor 402 during execution thereof by the machine 400. In an example, one or any combination of the hardware processor 402, the main memory 404, the static memory 406, or the storage device 416 may constitute machine readable media.

While the machine readable medium 422 is illustrated as a single medium, the term “machine readable medium” may include a single medium or multiple media (e.g., a centralized or distributed database, and/or associated caches and servers) configured to store the one or more instructions 424.

The term “machine readable medium” may include any medium that is capable of storing, encoding, or carrying instructions for execution by the machine 400 and that cause the machine 400 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine-readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 424 may turnier be transmitted or received over a communications network 426 using a transmission medium via the network interface device 420 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 420 may include one or more physical jacks (e.g., Ethernet, coaxial, or phone jacks) or one or more antennas to connect to the communications network 426. In an example, the network interface device 420 may include a plurality of antennas to communicate wirelessly using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 400, and includes digital or analog communications signals or other intangible medium to facilitate communication of such software.

Various embodiments of the present subject matter support wireless communications with a hearing device. In various embodiments the wireless communications may include standard or nonstandard communications. Some examples of standard wireless communications include link protocols including, but not limited to, Bluetooth™, Bluetooth™ Low Energy (BLE), IEEE 802.11 (wireless LANs), 802.15 (WPANs), 802.16 (WiMAX), cellular protocols including, but not limited to CDMA and GSM, ZigBee, and ultra-wideband (UWB) technologies. Such protocols support radio frequency communications and some support infrared communications while others support NFMI. Although the present system is demonstrated as a radio system, it is possible that other forms of wireless communications may be used such as ultrasonic, optical, infrared, and others. It is understood that the standards which may be used include past and present standards. It is also contemplated that future versions of these standards and new future standards may be employed without departing from the scope of the present subject matter.

The wireless communications support a connection from other devices. Such connections include, but are not limited to, one or more mono or stereo connections or digital connections having link protocols including, but not limited to 802.3 (Ethernet), 802.4, 802.5, USB, SPI, PCM, ATM, Fibre-channel, Firewire or 1394, InfiniBand, or a native streaming interface. In various embodiments, such connections include all past and present link protocols. It is also contemplated that future versions of these protocols and new future standards may be employed without departing from the scope of the present subject matter.

Hearing assistance devices typically include at least one enclosure or housing, a microphone, hearing assistance device electronics including processing electronics, and a speaker or “receiver.” Hearing assistance devices may include a power source, such as a battery. In various embodiments, the battery is rechargeable. In various embodiments multiple energy sources are employed. It is understood that in various embodiments the microphone is optional. It is understood that in various embodiments the receiver is optional. It is understood that variations in communications protocols, antenna configurations, and combinations of components may be employed without departing from the scope of the present subject matter. Antenna configurations may vary and may be included within an enclosure for the electronics or be external to an enclosure for the electronics. Thus, the examples set forth herein are intended to be demonstrative and not a limiting or exhaustive depiction of variations.

It is understood that digital hearing assistance devices include a processor. In digital hearing assistance devices with a processor, programmable gains may be employed to adjust the hearing assistance device output to a wearer's particular hearing impairment. The processor may be a digital signal processor (DSP), microprocessor, microcontroller, other digital logic, or combinations thereof. The processing may be done by a single processor, or may be distributed over different devices. The processing of signals referenced in this application may be performed using the processor or over different devices. Processing may be done in the digital domain, the analog domain, or combinations thereof. Processing may be done using subband processing techniques. Processing may be done using frequency domain or time domain approaches. Some processing may involve both frequency and time domain aspects. For brevity, in some examples drawings may omit certain blocks that perform frequency synthesis, frequency analysis, analog-to-digital conversion, digital-to-analog conversion, amplification, buffering, and certain types of filtering and processing. In various embodiments of the present subject matter the processor is adapted to perform instructions stored in one or more memories, which may or may not be explicitly shown. Various types of memory may be used, including volatile and nonvolatile forms of memory. In various embodiments, the processor or other processing devices execute instructions to perform a number of signal processing tasks. Such embodiments may include analog components in communication with the processor to perform signal processing tasks, such as sound reception by a microphone, or playing of sound using a receiver (i.e., in applications where such transducers are used). In various embodiments of the present subject matter, different realizations of the block diagrams, circuits, and processes set forth herein may be created by one of skill in the art without departing from the scope of the present subject matter.

It is further understood that different hearing devices may embody the present subject matter without departing from the scope of the present disclosure. The devices depicted in the figures are intended to demonstrate the subject matter, but not necessarily in a limited, exhaustive, or exclusive sense. It is also understood that the present subject matter may be used with a device designed for use in the right ear or the left ear or both ears of the wearer.

The present subject matter is demonstrated for hearing devices, including hearing assistance devices, including but not limited to, behind-the-ear (BTE), in-the-ear (ITE), in-the-canal (ITC), receiver-in-canal (RIC), invisible-in-canal (IIC) or completely-in-the-canal (CIC) type hearing assistance devices. It is understood that behind-the-ear type hearing assistance devices may include devices that reside substantially behind the ear or over the ear. Such devices may include hearing assistance devices with receivers associated with the electronics portion of the behind-the-ear device, or hearing assistance devices of the type having receivers in the ear canal of the user, including but not limited to receiver-in-canal (RIC) or receiver-in-the-ear (RITE) designs. The present subject matter may also be used in hearing assistance devices generally, such as cochlear implant type hearing devices. The present subject matter may also be used in deep insertion devices having a transducer, such as a receiver or microphone. The present subject matter may be used in bone conduction hearing devices, in some embodiments. The present subject matter may be used in devices whether such devices are standard or custom fit and whether they provide an open or an occlusive design. It is understood that other hearing devices not expressly stated herein may be used in conjunction with the present subject matter.

This application is intended to cover adaptations or variations of the present subject matter. It is to be understood that the above description is intended to be illustrative, and not restrictive. The scope of the present subject matter should be determined with reference to the appended claims, along with the full scope of legal equivalents to which such claims are entitled.

What is claimed is:

1. A hearing device, comprising:
a housing;
a first sensor on or in a first side of the housing;
a second sensor on or in a second side of the housing; and
hearing electronics within the housing configured to be connected to a microphone, the hearing electronics including at least one processor and a memory including instructions that, when executed by the at least one processor, cause the at least one processor to perform operations to:
obtain a first temperature measurement from the first sensor;
obtain a second temperature measurement from the second sensor;
compare the first temperature measurement to the second temperature measurement;
based on the comparison, determine whether the hearing device is positioned on a right side or a left side of a wearer of the hearing device; and
configure the hearing device as a right device or a left device based on the determination.

2. The device of claim 1, wherein, to configure the hearing device, the at least one processor is configured to determine gain settings for the hearing device based on audiograms for left and right ears of the wearer.

3. The device of claim 1, wherein the at least one processor is further configured to:
determine a body temperature of the wearer using the first temperature measurement or the second temperature measurement.

4. The device of claim 3, wherein the at least one processor is further configured to:
send a notification to the wearer when the first temperature measurement or the second temperature measurement indicates an abnormal body temperature of the wearer.

5. The device of claim 4, wherein the notification to the wearer is delivered via a mobile application on a smartphone in communication with the hearing device.

6. The device of claim 5, wherein the notification to the wearer includes a message with instructions for reducing temperature in the hearing device.

7. The device of claim 4, wherein the notification to the wearer is delivered via the hearing device.

8. The device of claim 7, wherein the notification to the wearer includes an audible notification.

9. The device of claim 1, wherein the memory includes firmware for the device.

10. The device of claim 1, wherein the hearing device is a hearing aid.

11. A method, comprising:
obtaining a first temperature measurement from a first sensor from a first side of a housing of a hearing device;
obtaining a second temperature measurement from a second sensor from a second side of the housing;
comparing the first temperature measurement to the second temperature measurement;
based on the comparison, determining whether the hearing device is positioned on a right side or a left side of a wearer of the hearing device; and
configuring the hearing device as a right device or a left device based on the determination.

12. The method of claim 11, wherein configuring the hearing device includes determining gain settings for the hearing device based on audiograms for left and right ears of the wearer.

13. The method of claim 11, wherein configuring the hearing device includes determining a stereo signal to stream to the hearing device.

14. The method of claim 11, wherein configuring the hearing device includes configuring controls on the housing of the hearing device.

15. The method of claim 11, further comprising:
sending a notification to the wearer when, based on the comparison, the hearing device is placed in a wrong ear of the wearer.

16. The method of claim 11, further comprising:
determining a body temperature of the wearer using the first temperature measurement or the second temperature measurement.

17. The method of claim 16, further comprising:
sending a notification to the wearer when the first temperature measurement or the second temperature measurement indicates an abnormal body temperature of the wearer.

18. The method of claim 11, further comprising:
automatically turning the hearing device on when the first temperature measurement or the second temperature measurement indicates that the hearing device has been placed on an ear of the wearer.

19. The method of claim 11, further comprising:
automatically turning the hearing device off when the first temperature measurement or the second temperature measurement indicates that the hearing device has been removed from an ear of the wearer.

20. The method of claim 11, further comprising:
automatically disconnecting a battery of the hearing device when the first temperature measurement or the second temperature measurement indicates a high temperature event above a predetermined temperature threshold.
